(19) United States
(12) Patent Application Publication
(10) Pub. No.: US 2007/0131342 A1
(43) Pub. Date: Jun. 14, 2007

(54) Method of making, using and applying a composition to an exposed surface

(75) Inventors: Chantel Spring Buhrow, Weyauwega, WI (US); Bernard Joseph Minerath III, Oshkosh, WI (US); Chelsea Marie Magin, Gainesville, FL (US)

Correspondence Address: KIMBERLY-CLARK WORLDWIDE, INC., 401 NORTH LAKE STREET, NEENAH, WI, 54956, US

(73) Assignee: Kimberly-Clark Worldwide, Inc.

(21) Appl. No.: 11/297,674

(22) Filed: Dec. 8, 2005

(51) Int. Cl. A61F 13/15

(52) U.S. Cl. 156/227; 134/6; 604/290

(57) ABSTRACT

Methods of making, using and applying a composition to an exposed surface are disclosed using a disposable applicator. The method of making the disposable applicator includes forming a back sheet and a liquid permeable substrate. The substrate is secured to the back sheet and a composition is applied to the substrate. The composition is capable of being transferred onto an exposed surface by wiping the substrate against the exposed surface. A seal is formed on at least a portion of the back sheet. The back sheet is then folded upon itself such that the seal extends completely around the substrate to protect the composition. The methods of using and applying a composition to an exposed surface include breaking the seal and opening the back sheet, grasping the back sheet in one hand and wiping the substrate onto the exposed surface.

### BACKGROUND OF THE INVENTION

Presently, there is a need for a sanitary disposable applicator which can be used to apply a composition onto an exposed surface and then be discarded. One application for such a disposable applicator is when a mother wishes to apply an ointment or cream directly onto a baby's buttock for the treatment of a diaper rash. Up until now, the mother could apply the baby rash ointment or cream directly onto the baby's buttock using her fingers. However, this is messy especially when performed at a location away from proper washing facilities. In addition, it can be very difficult for a mother to evenly apply the ointment or cream over the affected area of the skin. An alternative is for the mother to apply the baby rash ointment or cream onto the inside surface of the infant's diaper. However, some mothers do not feel confident that a sufficient quantity of the ointment or cream will transfer to the baby's skin in the area where the diaper rash is present. Another application of a disposable applicator is for applying medication onto a sore or wound formed in a person's skin. Still other uses for disposable applicators are to apply a variety of different compositions, for example sunscreen, furniture polish, etc., onto exposed surfaces. The surfaces can be a human body, the skin of an animal or the surface of an inanimate object.

Now a disposable applicator has been invented that can be used to transfer a composition, such as a skin care product, onto an exposed surface, for example, a baby's buttock. The disposable applicator can be discarded after a single use.

### SUMMARY OF THE INVENTION

Briefly, this invention relates to a method of making, using and applying a composition or formulation to an exposed surface using a disposable applicator. The method of making a disposable applicator includes forming a back sheet with an inner surface, an outer surface and a perimeter. The method also includes forming a liquid permeable substrate having a first surface, a second surface and a perimeter. The first surface of the substrate is then secured to the inner surface of the back sheet. A composition or formulation is applied to the liquid permeable substrate. The composition is capable of being transferred to an exposed surface by wiping the liquid permeable substrate against an exposed surface. The method further includes forming a seal on the inner surface of the back sheet. The back sheet is then folded upon itself such that the seal extends completely around the perimeter of the substrate and seals the composition or formulation.

The method of using the disposable applicator to transfer a composition or formulation onto an exposed surface includes breaking the seal and opening the back sheet into a relatively flat or planar orientation. The user would then grasp the back sheet in one hand and position the liquid permeable substrate in contact with the exposed surface. The exposed surface can be human skin, the body of an animal, a plant, or the surface of an inanimate object. The disposable applicator is then wiped over the exposed surface such that the composition or formulation is transferred onto the exposed surface.

The method of applying a composition or formulation onto a human skin using a disposable applicator includes breaking the seal and opening the back sheet into a relatively flat or planar orientation. The user would then grasp the back sheet in one hand and position the liquid permeable substrate in contact with the human skin. The disposable applicator is then wiped over the human skin such that the composition or formulation is transferred onto the human skin.

### BRIEF DESCRIPTION OF THE DRAWINGS

FIG. 1 is a top view of a disposable applicator showing a liquid permeable substrate having a composition applied to it and being secured to a back sheet and having a seal formed on the inner surface of the back sheet which at least partially surrounds the perimeter of the substrate.

FIG. 2 is a right side view of the disposable applicator shown in FIG. 1.

FIG. 3 is a plane view of the disposable applicator shown in FIG. 1 after the back sheet has been folded approximately at the transverse axis Y-Y.

FIG. 4 is a cross-sectional view of FIG. 3 taken along line 4-4 showing a chamber formed within the seal.

FIG. 5 is a bottom view of the disposable applicator shown in FIG. 1 depicting a finger pocket formed by folding the back sheet upon itself and having an advertisement appearing on an opposite portion of the back sheet.

FIG. 6 is a cross-sectional view of FIG. 5 taken along line 6-6.

FIG. 7 is a top view of an alternative embodiment of a disposable applicator showing an oval shaped, liquid permeable substrate having a composition applied to it and being secured to a back sheet, and having a seal formed completely around the perimeter of the substrate.

FIG. 8 is a right side view of the disposable applicator shown in FIG. 7.

FIG. 9 is a bottom view of the disposable applicator shown in FIG. 7 depicting a finger pocket formed by attaching a separate member to the outer surface of the back sheet.

FIG. 10 is a plane view of the disposable applicator shown in FIGS. 7-9 after the back sheet has been folded approximately at the transverse axis Y-Y.

FIG. 11 is a top view of a disposable applicator having a diamond configuration.

FIG. 12 is a top view of the disposable applicator shown in FIG. 11 after it has been folded along the transverse axis Y-Y.

FIG. 13 is a flow diagram depicting a method of making a disposable applicator.

FIG. 14 is a flow diagram depicting a method of using a disposable applicator.

FIG. 15 is a flow diagram depicting a method of applying a composition to a human's skin using a disposable applicator.

### DETAILED DESCRIPTION

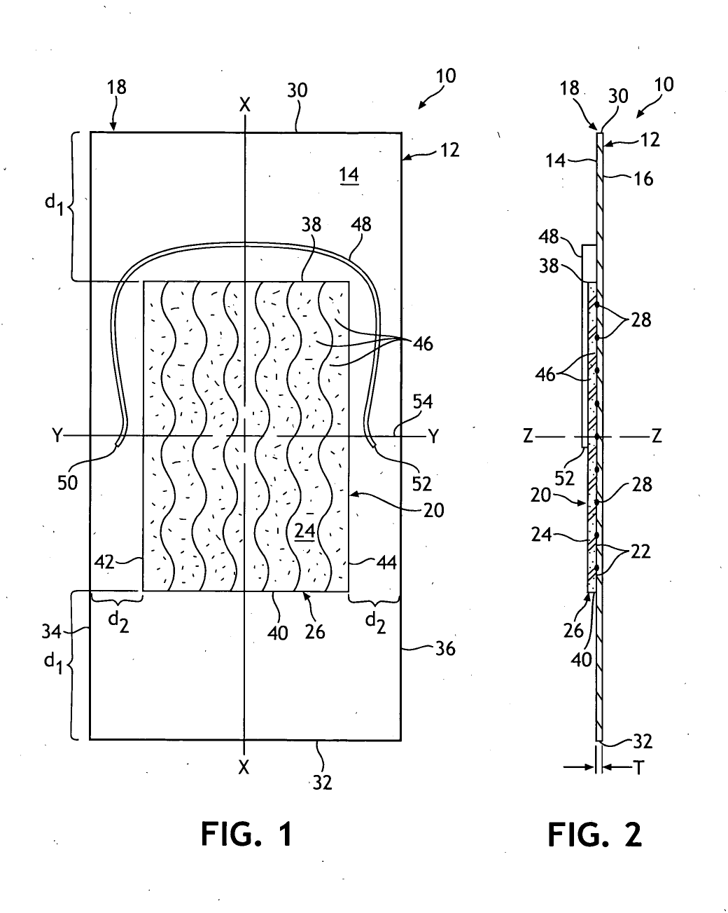

Referring to FIGS. 1 and 2, a disposable applicator 10 is shown having a longitudinal axis X-X, a transverse axis Y-Y, and a vertical axis Z-Z. The disposable applicator 10 includes a back sheet 12 having an inner surface 14 and an outer surface 16. The back sheet 12 also has a perimeter 18. Desirably, the back sheet 12 is liquid-impermeable. By “liquid-impermeable” it is meant that a fluid or liquid can not pass therethrough. More desirably, the back sheet 12 is moisture-impermeable. By “moisture-impermeable” it is meant that moisture can not pass therethrough. Even more desirably, the back sheet 12 is vapor-impermeable. By “vapor-impermeable” it is meant that a vapor, gas or air can not pass therethrough. A vapor is a gaseous state of a substance that is a liquid or solid under ordinary conditions. Air is a colorless, odorless, tasteless gaseous mixture, mainly nitrogen (approximately 78%) and oxygen (approximately 21%) with lesser amounts of argon, carbon dioxide, neon, helium, and other gases.

The back sheet 12 can be formed from a variety of materials, including natural and/or synthetic materials. Desirably, the back sheet 12 is a film, although a nonwoven material, a woven material or a combination of materials can be used. The back sheet 12 can be a film formed from a polyolefin, such as polyethylene or polypropylene. The back sheet 12 can also be formed from some other thermoplastic film or be a laminate of two or more layers wherein at least one layer is a barrier layer. As used herein, the term “laminate” refers to a composite structure of two or more material layers that have been adhered or otherwise bonded together, such as through adhesive bonding, thermal bonding, point bonding, pressure bonding, extrusion coating, ultrasonic bonding or by some other bonding technique known to those skilled in the art.

The back sheet 12 can include one or more barrier layers that can be in the form of a film layer. Furthermore, the back sheet 12 can be formed of a metallized material, such as a metal foil. A metal is any of a category of electropositive elements that are usually whitish, lustrous, and, in the transition metals, typically ductile and malleable with high tensile strength. A thin sheet of aluminum can also be used to form the back sheet 12. These and other specific examples of certain materials that work well include: a low density polyethylene (LDPE), such as a LDPE/SURLYN film, and metallized polyethylene terephthalate (MPET), which is a metallized film laminate. SURLYN is a registered trademark of E.I. Du Pont De Nemours and Company, having an office at 1007 Market Street, Wilmington, Del. 19898. These and other materials known to those skilled in the art can be used to form the back sheet 12.

The outer surface 16 of the back sheet 12 can also be made or constructed to have a rough or coarse texture or to have a grainy, fibrous or abrasive surface. This rough texture is the opposite of a smooth, slick or flat surface, as serves to limit slipping when the disposable applicator 10 is held within one's hand or between a person's thumb and one or more of their fingers. The method of using the disposable applicator 10 will be explained later.

Referring to FIG. 2, the back sheet 12 has a thickness “t” that is relatively thin. The thickness “t” of the back sheet 12 can be less than about 3 millimeters. Desirably, the thickness “t” of the back sheet 12 can be less than about 1.0 millimeter. Most desirably, the thickness “t” of the back sheet 12 can be less than about 0.1 millimeters. The backsheet 12 should also be flexible and have a soft feel to the hand. The back sheet 12 can be colored or have no color. A white color normally conveys a clean and sanitary appearance and is desirable for a disposable applicator 10 that is designed to come in contact with the human skin.

The back sheet 12 can be formed from a variety of water soluble or dispersible natural and synthetic polymers/film forming materials or combinations including but not limited to: PVP based polymers (e.g., polyvinyl pyrroidone), polyvinyl alcohol, polyethylene glycol (polyox), xantham gum, polyquaternium polymers, pullulan, hydroxypropylmethyl cellulose, hydroxyethyl cellulose, hydroxypropyl cellulose gelatin, carboxymethyl cellulose, polyvinyl alcohol, sodium alginate, tragacanth gum, guar gum, acacia gum, Arabic gum, polyacrylic acid, methylmethacylate copolymer, carboxyvinyl polymer, amylase, high amylase starch, hydroxypropylated high amylase starch, dextrin, pectin, chitin, chitosan, levan, elsinan, collagen, zein, glutan, soy protein isolate, whey protein isolate, casein and mixtures thereof.

The back sheet 12 can also be manufactured to be environmentally friendly. For example, the back sheet 12 can be made biodegradable. By “biodegradable” it is meant that the back sheet 12 is capable of being decomposed by natural biological processes. Illustrative examples include, but are not limited to, one or more of the following polymers: polyhydroxyalkonates, polylactic acid, and polyglycolide.

Still referring to FIGS. 1 and 2, the disposable applicator 10 also includes a substrate 20 having a first surface 22 and a second surface 24. The substrate 20 also has a perimeter 26, see FIG. 1. The perimeter 26 of the substrate 20 can be equal to or be smaller than the perimeter 18 of the back sheet 12. Desirably, the perimeter 26 of the substrate 20 is at least about 50% smaller than the perimeter 18 of the back sheet 12. More desirably, the perimeter 26 of the substrate 20 is at least about 25% smaller than the perimeter 18 of the back sheet 12. Even more desirably, the perimeter 26 of the substrate 20 is at least about 5% smaller than the perimeter 18 of the back sheet 12.

Referring again to FIG. 2, the first surface 22 of the substrate 20 is secured to the inner surface 14 of the back sheet 12 by an attachment 28. The attachment 28 can be a hot melt adhesive, a cold melt adhesive, a cohesive, a glue, an ultrasonic bond, a pressure bond, a thermal bond, a fusion bond, a heat and/or pressure bond, a chemical bond, a mechanical fastener, such as a hook and loop fastener, or by any other means known to those skilled in the art. Desirably, the attachment 28 is formed by an adhesive that can be applied as a continuous line or bead. Alternatively, the adhesive can be applied as intermittent spots, in a swirl pattern or in some other unique pattern or design. The adhesive can be applied in any of a variety of ways using commercially available equipment, spray heads, nozzles, etc. The first surface 22 of the substrate 20 is secured to the inner surface 14 of the back sheet 12 such that the inner surface 14 is coterminuous with the perimeter 26 of the substrate 20. Desirably, the inner surface 14 of the back sheet 12 extends completely around the perimeter 26 of the substrate 20. More desirably, the inner surface 14 of the back sheet 12 extends outward at least about 5 millimeters or more from the perimeter 26 of the substrate 20.

Referring again to FIG. 1, it has been found that when the back sheet 12 has a geometrical (i.e. rectangular, square, etc.) configuration with spaced apart ends 30 and 32, and spaced apart sides 34 and 36, and when the substrate 20 also has a geometrical (i.e. rectangular, square, etc.) configuration with spaced apart ends 38 and 40, and spaced apart sides 42 and 44, that the distance d₁ between the ends 38 and 40 of the substrate 20 and the adjacent ends 30 and 32 of the back sheet 12 should be equal to or be greater than the distance d₂ between the sides 42 and 44 of the substrate 20 and the adjacent sides 34 and 36 of the back sheet 12. This spacing is advantageous for it provides adequate surface area on the inner surface 14 of the back sheet 12 that can be used by a person to hold, retain and/or pinch the back sheet 12 between his or her thumb and one or more fingers in order to hold and move the disposable applicator 10 during use. More will be mentioned about this feature later.

The substrate 20 can be formed from a variety of materials that are liquid permeable or that are treated or coated so as to be liquid permeable. By “liquid permeable” it is meant that a liquid can penetrate through all or part of the material. The substrate 20 can be made from biodegradable materials such as cellulose, chitin, etc. The substrate 20 can be viewed as a delivery member that is capable of receiving a composition or formulation 46, retaining or holding the composition or formulation 46 for a period of time, and then at a later or prescribed time releasing or giving up the composition or formulation 46 to another surface or member. The substrate 20 can be a nonwoven web. As used herein, the term “nonwoven web” means a web having a structure of natural, synthetic or admixtures of natural and synthetic, fibers or threads which are interlaid, but not in an identifiable, repeating manner. Nonwoven webs can be formed by a variety of processes, such as a meltblown process, a spunbond process, a hydroentangling process, a wet-laid process, an air-laid process, a bonded carded web process, etc.

The substrate 20 can also be a bonded carded web. As used herein, the term “bonded carded web” refers to a web that is made from staple fibers which are usually purchased in bales. The bales are placed in a fiberizing unit/picker which separates the fibers. Next, the fibers are sent through a combining or carding unit which further breaks apart and aligns the staple fibers in a machine-direction direction so as to form a machine-direction-oriented fibrous nonwoven web. Once the web has been formed, it is then bonded by one or more of several bonding methods. One bonding method is powder bonding wherein a powdered adhesive is distributed throughout the web and then activated, usually by heating the web and adhesive with hot air. Another bonding method is pattern bonding wherein heated calender rolls or ultrasonic bonding equipment is used to bond the fibers together, usually in a localized bond pattern through the web, and/or alternatively, the web may be bonded across its entire surface, if so desired. When using bicomponent staple fibers, through-air bonding equipment is, for many applications, especially advantageous.

The substrate 20 can also be a spunbond, a spunbond laminate (SBL) or a bicomponent spunbond. As used herein, the term “spunbond” refers to small diameter fibers which are formed by extruding molten thermoplastic material as filaments from a plurality of fine, usually circular capillaries of a spinneret, with the diameter of the extruded filaments being rapidly reduced, such as by methods and apparatuses shown, for example, in U.S. Pat. No. 4,340,563 to Appel et al., and U.S. Pat. No. 3,692,618 to Dorschner et al., U.S. Pat. No. 3,802,817 to Matsuki et al., U.S. Pat. No. 3,338,992 to Kinney, U.S. Pat. No. 3,341,394 to Kinney, and U.S. Pat. No. 3,542,615 to Dobo et al., each of which is incorporated by reference and made a part hereof.

Furthermore, the substrate 20 can be a meltblown. As used herein, the term “meltblown” means fibers formed by extruding a molten thermoplastic material through a plurality of fine, usually circular die capillaries as molten threads or filaments into converging high velocity gas (i.e. air) streams which attenuate the filaments of molten thermoplastic material to reduce their diameter. The diameter of the meltblown may be reduced to microfiber diameter. Thereafter, the meltblown fibers are carried by the high velocity gas stream and are deposited on a collecting surface to form a web of randomly dispersed meltblown fibers. Such a process is disclosed in various patents and publications, including NRL Report 4364, “Manufacture of Super-Fine Organic Fibers” by B. A. Wendt, E. L. Boone and D. D. Fluharty; NRL Report 5265, “An Improved Device For The Formation of Super-Fine Thermoplastic Fibers” by K. D. Lawrence, R. T. Lukas, J. A. Young; and U.S. Pat. No. 3,849,241, issued Nov. 19, 1974, to Butin, et al., which are incorporated by reference hereto in their entirety.

The substrate 20 can further be formed from HYDROKNIT. HYDROKNIT is a high pulp content hydraulically entangled nonwoven composite fabric which is taught and described in U.S. Pat. No. 5,284,703. A process for making HYDROKNIT is taught and described in U.S. Pat. No. 5,389,202. Both of these patents are incorporated by reference and made a part hereof. HYDROKNIT is a registered trademark of Kimberly-Clark Corporation, having an office at 401 North Lake Street, Neenah, Wis. 54956. The substrate 20 can also be formed from an airlaid nonwoven, a coform, or a variety of other materials known to those skilled in the art. Airlaid nonwoven is fabric made by an air-forming process wherein the fibers are distributed by air currents to give a random orientation within the web and the fabric has isotropic properties. Coform is a material made from a blend of wood pulp fibers and polypropylene fibers. Airlaid and coform materials are commercially available from a variety of vendors.

The substrate 20 can be constructed from one or more layers that are arranged in a vertical or horizontal fashion. By “vertical” it is meant that one layer is positioned vertically above another layer. By “horizontal” it is meant that one layer is positioned to the side of another layer; for example, substrate “A” next to substrate “B”. In the horizontal arrangement, each substrate does not have to be constructed of similar or identical materials.

The composition or formulation 46 is applied to the liquid permeable substrate 20. The composition or formulation 46 impregnates the structure of the liquid permeable substrate 20 and is thereby temporarily retained therein. By “temporary” it is meant that the composition or formulation 46 can be held or retained by the substrate until needed. This time period can be one or more seconds, minutes, hours, days, weeks, months or even years. The composition or formulation 46 is of such a consistency and characteristic that it is capable of being transferred from the substrate 20 onto an exposed surface by wiping or moving the liquid permeable substrate 20 against the exposed surface. The exposed surface can be an area or part of a human body, such as the skin or hair. The exposed surface can also be an area or part of an animal's body, including its skin, hair or fur. In addition, the exposed surface can be an area or part of a natural plant, flower, tree, etc. Furthermore, the exposed surface can be an inanimate object, such as a piece of furniture, the hood of a car, an eating utensil, a fabric, carpeting, clothing, etc.

The composition or formulation 46 can be a variety of items including but not limited to aqueous solutions, ointments, creams, emulsions, solvents, etc. The composition or formulation 46 can be any of a variety of skin treatments that can be delivered in effective amounts from the substrate 20 to a human's skin. Such skin treatments include but are not limited to alpha and/or beta hydroxyl-acids, analgesics, antimicrobials, antibodies, anti-acne, anti-irritants, antigens,anesthetics, anti-inflammatory substances, antioxidants, anti-adherents, anti-pruritic materials, astringents, botanicals, botanical extracts, buffers, chelators, cleansing compositions, emollients, exfolliants, proteins, lipids, carbohydrates, derivatives of each of the previous three items, enzymes, enzyme inhibitors, growth factors, irritant sequestrants, skin lightening agents, hair care compositions, depilatory compositions, shaving aids, fragrances, metal salts, microbial growth factors, microorganisms, odor absorbents, oxidizers, probiotics, moisturizers, color cosmetics, vasoconstrictors, muscle relaxants, lubricants, sunscreen, insect repellents, skin protectants, surfactants, vitamins, volatile organic compound (VOC) absorbents, material to alter skin and/or hair surface energy, and the like could be delivered alone or in any number of permutations or combinations.

The composition or formulation 46 can also be in the physical form of a liquid, a powder, a lotion, a gel, a cream, an emulsion, a soft solid, a semi-solid and even a solid. The composition or formulation 46 can be a liquid, a semi-solid or a solid at a certain temperature, for example at 70 degrees Fahrenheit (F°). The composition or formulation 46 can be a Newtonian fluid or a Non-Newtonian fluid. A Newtonian fluid is a fluid in which the viscosity does not change as the applied shear force changes. A Non-Newtonian fluid is a fluid in which the viscosity changes with the applied shear force. As a result, Non-Newtonian fluids may not have a well-defined viscosity. The composition or formulation 46 can be a medicament, a petroleum gel, a highly viscous product, an ointment having a gel-like consistency, etc.

It has been found that for many skin care compositions or formulations that they tend to work best when they are homogeneous. In addition, it is important that a skin care composition or formulation rapidly de-emulsify upon application to the skin and that it does not re-emulsify. Furthermore, for a skin care composition or formulation it should also be manufactured such that it has the ability to inhibit the growth of a broad range of bacteria, fungi and other microorganisms.

The composition or formulation 46 can include one or more additives, such as a lubricant which can reduce abrasion when the skin, a colorant to impart a particular color, or a fragrant which can provide a pleasant odor. These and other additives known to those skilled in the art can be utilized.

It should also be understood that any of a variety of non-skin care compositions or formulations can be delivered from the substrate 20 of the disposable applicator 10. These include but are not limited to hard surface disinfectants, ultraviolet (UV) protectants, sun screens, sanitizers, polymer conditioners, polishes, scrubbing agents, cleansing solutions, cleansers, degreasers, adhesives, lubricants, soil repellents, water repellents, pesticides, insect repellents, pest baits, bleaching agents, antimicrobials, acids, bases, fragrances, pesticides, and the like. For some non-skin care compositions or formulations, they do not have to be homogeneous or exhibit the above-identified features desirable in a skin care composition or formulation.

The amount of the composition or formulation 46 that is applied to or impregnated into the substrate 20 can vary depending on the physical size of the substrate 20, the material from which it is constructed, the chemical makeup of the composition or formulation 46, the physical nature of the composition or formulation 46, the temperature at which the disposable applicator 10 is stored, etc. However, as an example, when the disposable applicator 10 is designed to deliver diaper rash cream or ointment, a sufficient quantity should be applied to the substrate 20 such that from between about 0.2 grams to about 2 grams of skin care composition or formulation can be delivered to a person's skin. Desirably, at least one gram of skin care composition or formulation will be delivered to a person's skin. When the composition or formulation is a diaper rash cream, more than one gram of the cream should be applied to the substrate 20 to ensure that at least one gram of the diaper rash cream will transfer onto the baby's skin. The actual amount of the composition or formulation that has to be applied to the substrate 20 will depend upon a number of factors, including the intended use of the formulation, the viscosity of the cream, if it is homogeneous, if it is a semi-solid at room temperature, etc. The diaper rash cream is a topical protective barrier agent that is applied to the baby's skin, i.e. the buttocks, and acts between the skin and any body waste to prevent the skin from being harmed.

A variety of vehicles for delivering any one of the above-identified composition or formulation 46 from the substrate 20 of the disposable applicator 10 are envisioned. These include but are not limited to ointments, macro, micro and nano emulsions, creams, suspensions, solutions and the like. The vehicles may contain water but anhydrous vehicles are also envisioned. In certain embodiments, these vehicles might also include encapsulation technologies, such as liposomes, colloidosomes, coated particulates and other methods known to those skilled in the art. Application of the composition or formulation 46 to any anatomical site on the human body, to the body of an animal, or to an inanimate object is envisioned.

Referring again to FIGS. 1 and 2, the disposable applicator 10 further includes a seal 48 that is formed on the inner surface 14 of the back sheet 12. The seal 48 can be a breakable seal or a non-breakable seal. Desirably, the seal 48 is a breakable seal. The seal 48 can be formed by applying one or more sealing technologies, such as adhesive, cohesive, glue, an ultrasonic bond, a chemical bond, a thermal bond, a fusion bond, a heat and pressure bond, etc., to the inner surface 14 of the back sheet 12. Desirably, the seal 48 is an adhesive bond. The seal 48 should be positioned at least about 10 millimeters inward from the perimeter 18 of the back sheet 12. Desirably, the seal 48 should be positioned at least about 5 millimeters inward from the perimeter 18 of the back sheet 12. More desirably, the seal 48 should be positioned at least about 1 millimeter outward from the perimeter 26 of the substrate 20. Even more desirably, the seal 48 should be positioned from between about 1 to about 5 millimeters outward from the perimeter 26 of the substrate 20. Still more desirably, the seal 48 is positioned from at least about 3 millimeters outward from the perimeter 26 of the substrate 20.

In FIGS. 1 and 2, the seal 48 is a continuous line of adhesive that has first and second ends 50 and 52. The thickness and height of the seal 48 can vary depending on the size, height and configuration of the substrate 20. The seal 48 extends approximately half way around the perimeter 26 of the substrate 20 and on one side of the transverse axis Y-Y. The seal 48 actually extends at least about 50% around the perimeter 26 of the substrate 20 and crosses the transverse axis Y-Y. The reason for this configuration is that as the back sheet 12 is folded upon itself at a fold line 54, that is aligned approximately with the transverse axis Y-Y, the seal 48 will attach to the remaining portion of the inner surface 14 and thereby extend completely around the perimeter 26 of the substrate 20. This embodiment saves on the amount of adhesive that has to be used to form the seal 48. The seal 48 functions to prevent the composition or formulation 46 that has been applied to the substrate 20 from escaping or leaking out and also prevents air, moisture and possible contaminants from getting into the substrate 20 and somehow detrimentally affecting the composition or formulation 46.

The ability to seal 48 to form an air permeable envelope around the substrate 20 allows one to maintain the stability of certain compositions or formulations 46 such as anti-oxidants and anaerobic bacteria. In addition, one can maintain the stability of other oxygen labile composition or formulations 46.

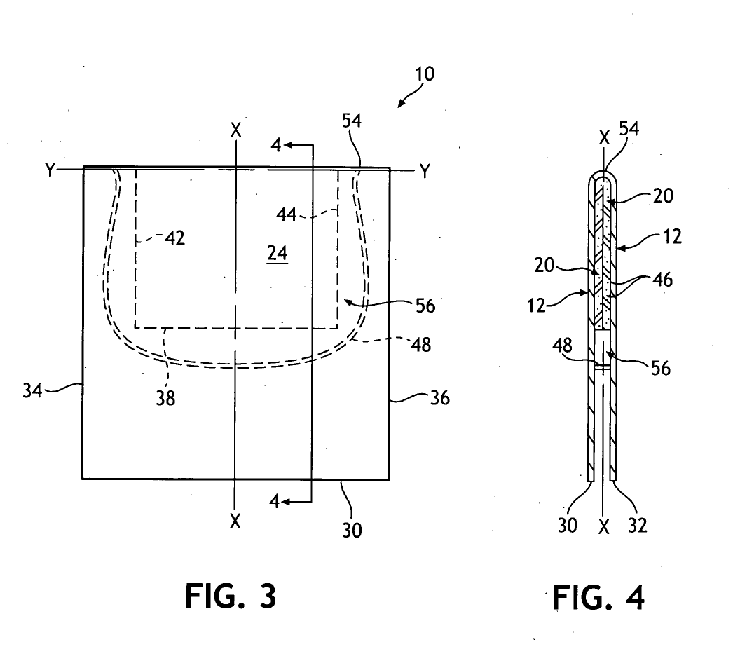

Referring now to FIGS. 3 and 4, the seal 48 provides a temporary seal in the sense that it is designed to be broken when one pulls on the opposite ends 30 and 32 of the back sheet 12. This pulling action will break the seal 48 and allow the back sheet 12 to be opened into a flat or planar orientation, such as is depicted in FIG. 1. When the back sheet 12 is folded upon itself along the fold line 54, a chamber or cavity 56 is formed around the substrate 20 which encloses the substrate and composition or formulation applied thereto. In FIG. 4, one can see that the ends 30 and 32 of the back sheet 12 are separated slightly on the outside of the seal 48. This separation facilitates easy opening of the back sheet 12 by allowing the user to grasp each end 28 and 30 and pull the two ends 28 and 30 apart. This pulling action will cause the seal 48 to break or separate from itself and/or the inner surface 14 of the back sheet 12. As this occurs, the chamber or cavity 56 will be opened and the substrate 20 with its applied composition or formulation 46 will be exposed. The back sheet 12 should be opened fully to its flat or planar configuration, i.e. its original configuration before it was folded.

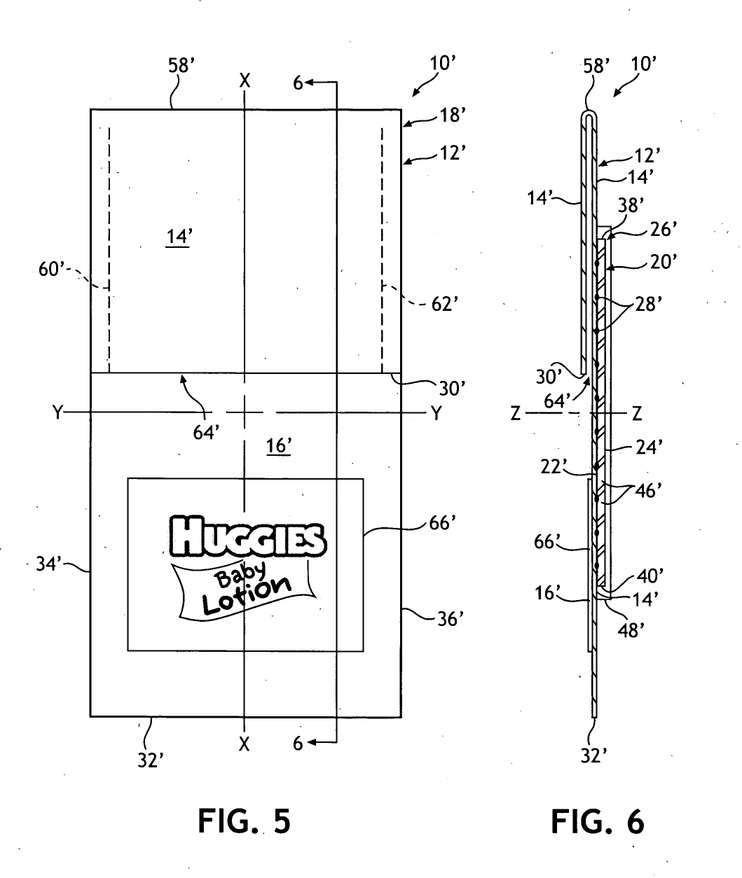

Referring to FIGS. 5 and 6, an alternative embodiment of a disposable applicator 10′ is shown. Similar numbers as used in FIGS. 1-4 will be used for common elements along with the prime symbol (′) to simplify understanding of the invention. In FIG. 5, the disposable applicator 10′ is shown having a back sheet 12′ with an inner surface 14′ and an outer surface 16′. The back sheet 12′ is longer in length than the backsheet 12 that is shown in FIGS. 1-4. The back sheet 12′ also has a perimeter 18′. A substrate 20′, see FIG. 6, includes a first surface 22′ and a second surface 24′. The substrate 22′ also has a perimeter 26′. The first surface 22′ of the substrate 20′ is attached to the first surface 14′ of the back sheet 12′ by an attachment 28′. The attachment 28′ can be an adhesive or any of the other attachment means recited above with reference to FIGS. 1-4. The back sheet 12′ has spaced apart ends 30′ and 32′ and spaced apart sides 34′ and 36′. The substrate 20′ also has spaced apart ends 38′ and 40′, see FIG. 6, and spaced apart sides that are not visible in FIGS. 5 or 6. A composition or formulation 46′ is applied to the liquid permeable substrate 20′. The disposable applicator 10′ further includes a seal 48′ that is formed on the inner surface 14′ of the back sheet 12′. The seal 48′ can be a breakable seal or a non-breakable seal. Desirably, the seal 48′ is a breakable seal. The seal 48′ can be formed by applying adhesive, cohesive, glue, an ultrasonic bond, a chemical bond, a thermal bond, a fusion bond, a heat and pressure bond, etc., to the inner surface 14′ of the back sheet 12′. Desirably, the seal 48′ is an adhesive bond. Contrary to the seal 48 shown in FIGS. 1 and 2, the seal 48′ in FIG. 6 extends completely around the perimeter 26′ of the substrate 20′. The seal 48′ is positioned around the perimeter 26′ of the substrate 20′. The seal 48′ is located from between about 1 to about 15 millimeters outward from the perimeter 26′ of the substrate 20′. More desirably, the seal 48′ is positioned at least about 3 millimeters outward from the perimeter 26′ of the substrate 20′. In FIG. 6 the seal 48′ is shown as a continuous line of adhesive.

One portion of the back sheet 12′ is folded at fold line 58′ upon itself such that the outer surface 16′ is folded and overlaps another section of the outer surface 16′ of the back sheet 12′. This folded portion has a pair of side edges 60′ and 62′ that are then sealed to form a pocket 64′, see FIGS. 5 and 6. The pocket 64′ is located between the outer surface 16′ of the back sheet 12′. The side edges 60′ and 62′ of the folded portion of the back sheet 12′ can be sealed or bonded using a hot melt adhesive, a cold melt adhesive, a cohesive, a glue, an ultrasonic bond, a pressure bond, a thermal bond, a fusion bond, a heat and pressure bond, a chemical bond, a mechanical fastener, such as a hook and loop fastener, or by any other means known to those skilled in the art. Desirably, the side edges 60′ and 62′ are sealed by an ultrasonic bond. The bonds forming the pocket 64′ should be stronger than the breakable seal 48′ so as to ensure that the pocket 64′ will not break should the user insert his or her fingers into the pocket 64′ while the disposable applicator 10′ is in a closed position and then use his or her fingers that are positioned within the pocket 64′ to assist in causing the breakable seal 48′ to be broken.

The pocket 64′ is sized and shaped to allow a user of the disposable applicator 10′ to insert at least one finger or digit of one of his or her hands comfortably into the pocket 64′. By “digit” it is meant a thumb. Desirably, two fingers and/or one digit of one hand can be inserted into the pocket 64′. More desirably, three or four fingers and/or a digit of one hand can be inserted into the pocket 64′. The function of the pocket 64′ is to allow the user to insert one, two, three or four fingers or digits into the pocket 64′ such that the user can move or wipe the substrate 20′ of the disposable applicator 10′ against an exposed surface. While control of the disposable applicator 10 or 10′ is best accomplished using the fingers, it is also possible for the user to accomplish this with any digit, including his or her thumb. The exposed surface can be his or her skin, the skin of another person, such as the skin of a child or older adult, the body of an animal, or the surface of an object. As the substrate 20′ is wiped against the exposed surface, the composition or formulation 46′, see FIG. 6, will be transferred from the substrate 20′ onto the exposed surface. It is anticipated that the user can apply a downward force or pressure onto the back sheet 12′ as the wiping occurs to assist the transfer of the composition or formulation 46′ onto the exposed surface.

The disposable applicator 10′ further has an advertisement 66′ printed, formed or attached to a remaining portion of the outer surface 16′ of the back sheet 12′. The advertisement 66′ can include letters, words, graphics symbols, icons, trademarks, instructions for use, safety instructions, instructions for proper disposal of the disposable applicator 10′, etc. The advertisement 66′ can be printed or embossed onto the outer surface 16′ of the back sheet 12′ or it can be in the form of a label or patch that is attached to the outer surface 16′ of the back sheet 12′. Various ways of printing, embossing, or constructing labels is known to those skilled in the art.

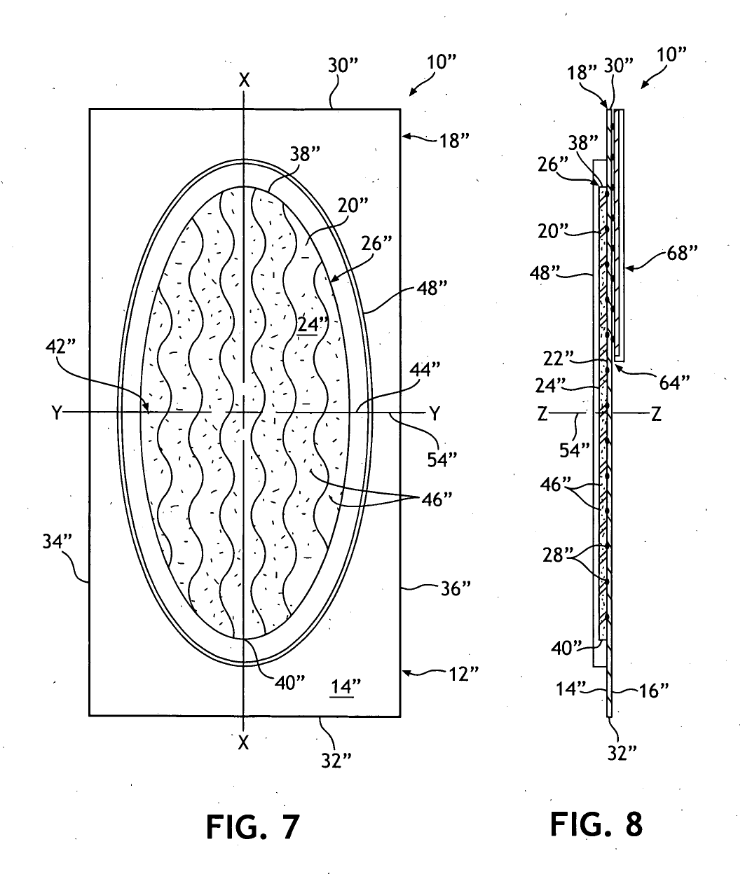

Referring now to FIGS. 7-10, another embodiment of a disposable applicator 10″ is depicted. Similar numbers as used in FIGS. 1-6 will be used for common elements along with the symbol (″) to simplify understanding of the invention. In FIG. 7, the disposable applicator 10″ is shown having a back sheet 12″ with an inner surface 14″ and an outer surface 16″. The back sheet 12″ is similar in length to the backsheet 12 that is shown in FIGS. 1-4. The back sheet 12″ also has a perimeter 18″. A substrate 20″ is also present, which includes a first surface 22″, see FIG. 8, and a second surface 24″. The substrate 22″ also has a perimeter 26″ best illustrated in FIG. 8. In FIG. 7, the substrate 20″ has an oval configuration as opposed to a rectangular configuration, shown in FIGS. 1-6. It should be noted that the substrate 20, 20′ and 20″ can have any desired geometrical shape. The first surface 22″ of the substrate 20″ is attached to the first surface 14″ of the back sheet 12″ by an attachment 28″. The attachment 28″ can be an adhesive or any of the other attachment means recited above with reference to FIGS. 1-6. The back sheet 12″ has spaced apart ends 30″ and 32″ and spaced apart sides 34″ and 36″. The substrate 20″ also has spaced apart ends 38′ and 40′, see FIG. 6, and spaced apart sides that are not visible in FIGS. 5 or 6. A composition or formulation 46″ is applied to the liquid permeable substrate 20″. The disposable applicator 10″ further includes a seal 48″ that is formed on the inner surface 14″ of the back sheet 12″. The seal 48″ can be a breakable seal or a non-breakable seal. Desirably, the seal 48″ is a breakable seal. The seal 48″ can be formed by applying adhesive, cohesive, glue, an ultrasonic bond, a chemical bond, a thermal bond, a fusion bond, a heat and pressure bond, etc., to the inner surface 14″ of the back sheet 12″. Desirably, the seal 48″ is an adhesive bond. Contrary to the seal 48 shown in FIGS. 1 and 2, the seal 48″ in FIGS. 7 and 8 extends completely around the perimeter 26″ of the substrate 20″. The seal 48″ is located from between about 1 to about 15 millimeters outward from the perimeter 26″ of the substrate 20″. Even more desirably, the seal 48″ is positioned at least about 3 millimeters outward from the perimeter 26″ of the substrate 20″. In FIGS. 7 and 8, the seal 48″ is shown as a continuous line of adhesive.

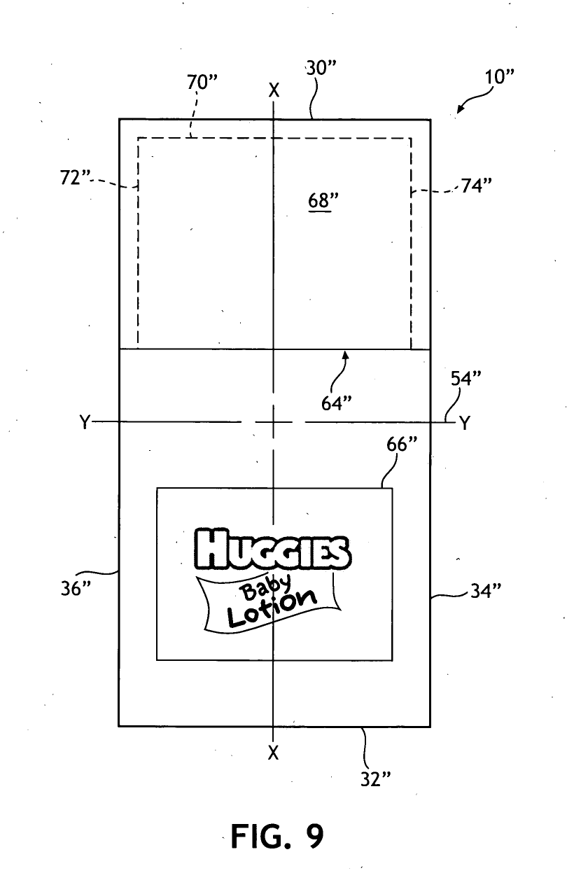

Referring to FIGS. 7-9, the disposable applicator 10″ also differs from the embodiment shown in FIGS. 5 and 6 in that it has a pocket 64″ formed by securing a separate piece of material 68″. The separate piece of material 68″ is secured to the outer surface 16″ of the back sheet 12″, see FIG. 7, by bond lines 70″, 72″ and 74″, see FIG. 9. The bond line 70″ is located adjacent to the end 30″ and the bond lines 72″ and 74″ are located adjacent to the sides 36″ and 34″, respectively. The bond lines 70″, 72″ and 74″ can be formed by using an adhesive, glue, by ultrasonic bonds or any of the other bonding mechanisms described above with relation to the attachments 28, 28′ and 28″. As stated above with reference to FIGS. 5 and 6, the bonds forming the pocket 64″ should be stronger than the breakable seal 48″ to ensure that the pocket 64″ does not come apart before the breakable seal 48″ breaks.

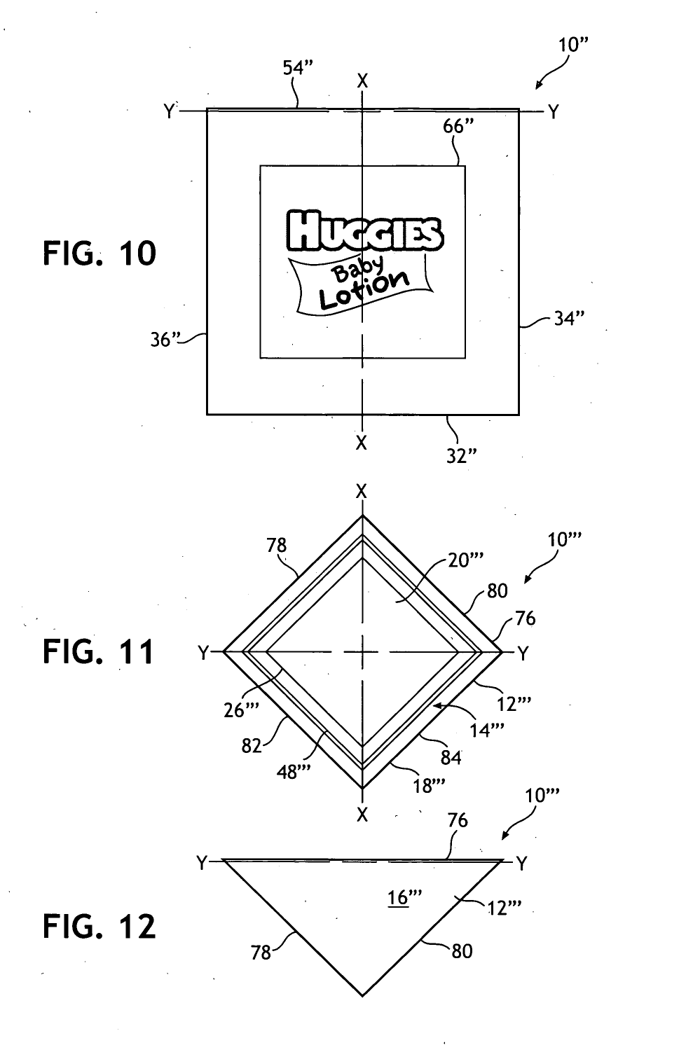

The disposable applicator 10″ also has a fold line 54″ aligned approximate with the transverse axis Y-Y, although it does not have to be so positioned. The disposable applicator 10″ is designed to be folded along the fold line 54″ in order to seal the substrate 20″ with the breakable seal 48″. Furthermore, the disposable applicator 10″ has an advertisement 66″ printed, formed or attached to a remaining portion of the outer surface 16″ of the back sheet 12″. The advertisement 66″ can include letters, words, graphics symbols, icons, trademarks, instructions for use, safety instructions, instructions for proper disposal of the disposable applicator 10″, etc. The advertisement 66″ can be printed or embossed onto the outer surface 16″ of the back sheet 12″ or it can be in the form of a label or patch that is attached to the outer surface 16″ of the back sheet 12″. Various ways of printing, embossing, or constructing labels is known to those skilled in the art. When the disposable applicator 10″ is folded along the fold line 54″ and the breakable seal 48″ is secure, the disposable applicator 10″ will acquire the configuration shown in FIG. 10.

Referring now to FIGS. 11 and 12, another embodiment of a disposable applicator 10′″ is shown having a diamond or square configuration with a longitudinal axis X-X and a transverse axis Y-Y. The disposable applicator 10′″ includes a back sheet 12′″ that has an inner surface 14′″, an outer surface 16′″ and a perimeter 18′″. The perimeter 18′″ includes sides 78, 80, 82 and 84. A substrate 20′″ is secured to the inner surface 14′″ of the back sheet 12′″ by any of the means described above. The substrate 20′″ has a perimeter 26′″ which is spaced inward from the perimeter 18′″, although the perimeters 18′″ and 26′″ can be coterminous, if desired. The disposable applicator 10′″ also includes a seal 48′″ formed outward of the perimeter 26′″ of the substrate 20′″. The seal 48′″ can be a breakable seal or a non-breakable seal. Desirably, the seal 48′″ is a breakable seal. The disposable applicator 10′″ is designed to be folded along a fold line 76. In FIG. 12, the fold line 76 is aligned along the transverse axis Y-Y, although it does not have to be. Once the disposable applicator 10′″ is folded along the fold line 76, the sides 78 and 80 will overlap the sides, 82 and 84 respectively. This creates a triangular configuration as depicted in FIG. 12. When the disposable applicator 10′″ is folded upon itself along the fold line 76, the seal 48′″ will adhere to either itself or to the inner surface 14′″ of the back sheet 12′″ along two sides. In FIG. 12, the seal 48′″ is aligned along the two sides 78 and 80 of the triangular configuration.

It should be recognized that the breakable seal 48′″ can be an adhesive that extends along at least two of the sides, 78 and 80 or 82 and 84, situated on one side of the fold line 76, similar to the breakable seal 48 shown in FIG. 1. Desirably, the breakable seal 48′″ will extend slightly beyond the fold line 76 to ensure that the folded disposable applicator 10′″ is sealed along the two sides that are aligned at an angle to the fold line 76. By positioning the breakable seal 48′″ on half or slightly more than half of the back sheet 12′″, one can save on the amount of adhesive that needs to be used in making the disposable applicator 10′″ and this can reduce the cost of the finished product.

It should also be recognized that any of the disposable applicators 10, 10′, 10″ or 10′″ can utilize an adhesive secured to the outer surface 16, 16′, 16″ or 16′″ of the respective backsheet 12, 12′, 12″ or 12′″. The adhesive can exhibit a residual tack. A label can then be removeably attached to the adhesive such that when the label is removed, the adhesive will enable a user to grasp the disposable applicator 10, 10′, 10″ or 10′″ in one hand and maintain enhanced control of the disposable applicator 10, 10′, 10″ or 10′″.

### METHOD

The methods of making a disposable applicator, using the disposable applicator, and applying a composition to an exposed surface with the disposable applicator will now be described.

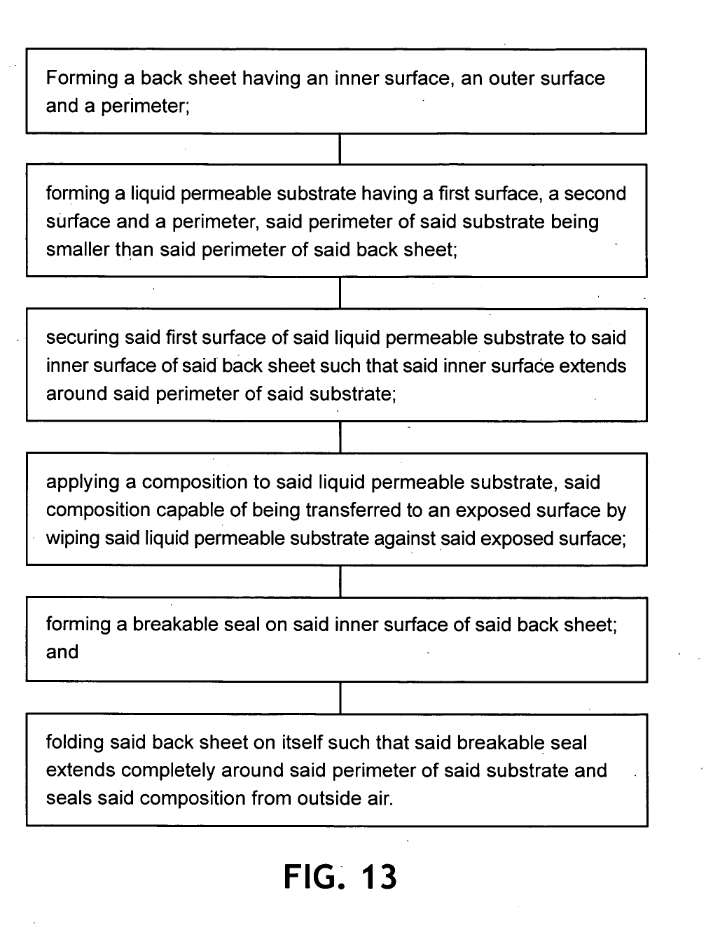

Referring to FIG. 13, a flow diagram is shown which depicts a method of making a disposable applicator 10, 10′, 10″ or 10′″. The disposable applicator 10, 10′, 10″ or 10′″ has a longitudinal axis X-X, a transverse axis Y-Y, and a vertical axis Z-Z. The method includes the steps of forming a back sheet 12,12′, 12″ or 12′″ having an inner surface 14, 14′, 14″ or 14′″, an outer surface 16, 16′, 16″ or 16′″ and a perimeter 18, 18′, 18″ or 18′″. The back sheet 12, 12′, 12″ or 12′″ can be formed from a nonwoven material having a narrow thickness. Desirably, the thickness of the back sheet 12, 12′, 12″ or 12′″ is less than about 3 millimeters. The back sheet 12, 12′, 12″ or 12′″ can be made vapor-impermeable, moisture-impermeable or liquid-impermeable. The decision to make the back sheet 12, 12′, 12″ or 12′″ vapor, moisture or liquid-impermeable will depend upon the composition or formulation 46, 46′ or 46″ that is applied to the substrate 20, 20′, 20″ or 20′″. A liquid permeable substrate 20, 20′, 20″ or 20′″ is also formed that has a first surface 22, 22′ or 22″, a second surface 24, 24′ or 24″ and a perimeter 26, 26′, 26″ or 26′″. The perimeter 26, 26′, 26″ or 26′″ of the substrate 20, 20′, 20″ or 20′″ respectively, is desirably smaller than the perimeter 18, 18′, 18″ or 18′″ of the back sheet 12, 12′, 12″ or 12′″ respectively.

The method includes securing the first surface 22, 22′ or 22″ of the liquid permeable substrate 20, 20′ or 20″ respectively, to the inner surface 14, 14′, 14″ or 14′″ of the back sheet 12,12′, 12″ or 12′″ respectively. The liquid permeable substrate 20, 20′, 20″ or 20′″ can be secured using an adhesive, glue, ultrasonics or any of the other means known to those skilled in the art. When the liquid permeable substrate 20, 20′, 20″ or 20′″ is secured or attached to the back sheet 12,12′, 12″ or 12′″ respectively, the inner surface 14, 14′, 14″ or 14′″ of the back sheet 12, 12′, 12″ or 12′″ respectively, extends around the perimeter 26, 26′, 26″ or 26′″ of the substrate 20, 20′ or 20″ respectively. The inner surface 14, 14′, 14″ or 14′″ of the back sheet 12, 12′, 12″ or 12′″ respectively, can extend completely around or be coterminous with the perimeter 26, 26′, 26″ or 26′″ of the respective substrate 20, 20′, 20″ or 20′″.

The method further includes applying a composition or formulation 46 to the liquid permeable substrate 20, 20′, 20″ or 20′″ respectively. The composition or formulation 46, 46′ or 46″ can be applied or coated onto a surface of the liquid permeable substrate 20, 20′, 20″ or 20′″ or it can penetrate and impregnate the liquid permeable substrate 20, 20′, 20″ or 20′″. The composition or formulation 46, 46′ or 46″ is capable of being transferred onto an exposed surface by wiping the liquid permeable substrate 20, 20′, 20″ or 20′″ against the exposed surface. The wiping action can be done at little or no pressure or at an applied pressure. For example, the liquid permeable substrate 20, 20′, 20″ or 20′″ can be wiped or moved over an exposed surface, such as human skin, while a pressure or applied force ranging from between about 0.01 pounds per square inch (psi) to about 5 psi is applied. The force or pressure can be applied by the user of the disposable applicator 10, 10′, 10″ or 10′″ or by a caregiver who is applying the composition or formulation 46, 46′ or 46″. Desirably, the wiping motion is accomplished while using an applied force ranging from between about 0.1 psi to about 3 psi. More desirably, the wiping motion is accomplished while using an applied force ranging from between about 0.2 psi to about 2 psi. Even more desirably, the wiping motion is accomplished while using an applied force ranging from between about 0.25 psi to about 1 psi.

The method further includes forming a seal 48, 48′, 48″ or 48′″, desirably a breakable seal, on the inner surface 14, 14′, 14″ or 14′″ of the back sheet 12, 12′, 12″ or 12′″ respectively. The seal 48, 48′, 48″ or 48′″ can be formed before or after the composition or formulation 46, 46′ or 46″ is applied to the liquid permeable substrate 20, 20′, 20″ or 20′″ respectively. The seal 48, 48′, 48″ or 48′″ can extend partially around the perimeter 18, 18′, 18″ or 18′″ of the substrate 20, 20′, 20″ or 20′″ respectively, as is depicted in FIG. 1. Alternatively, the seal 48, 48′, 48″ or 48′″ can extend completely around the perimeter 18, 18′, 18″ or 18′″ of the substrate 20, 20′, 20″ or 20′″ respectively, as is shown in FIGS. 7 and 11.

Still further, the method includes folding the back sheet 12, 12′, 12″ or 12′″ upon itself. The back sheet 12, 12′, 12″ or 12′″ can be folded either before or after the seal 48, 48′, 48″ or 48′″ is formed depending upon the structure of the respective disposable applicator 10,10′, 10″ or 10′″. The back sheet 12, 12′,12″ or 12′″ can be folded along its transverse axis or along the transverse axis Y-Y of the disposable applicator 10, 10′, 10″ or 10′″, see FIGS. 3, 4, 10 and 12. In FIGS. 3, 4, 10 and 11 the transverse axis of the back sheet 12, 12′, 12″ or 12′″ is the transverse axis Y-Y of the disposable applicator 10, 10′, 10″ or 10′″. When the back sheet 12, 12′, 12″ or 12′″ is folded upon itself, the seal 48, 48′, 48″ or 48′″ will extend completely around the perimeter 26, 26′, 26″ or 26′″ of the substrate 20, 20′, 20″ or 20′″. A chamber 56 is formed within an area encompassed by the seal 48, 48′, 48″ or 48′″ once the back sheet 12, 12′ 12″ or 12′″ is folded along the transverse axis Y-Y. The seal 48, 48′, 48″ or 48′″ functions to seal the composition or formulation 46 from outside air.

In FIGS. 6, 7, 8 and 11, the seal 48′, 48″ and 48′″ extends completely around the perimeter, 26′, 26″ or 26′″ respectively, of the liquid permeable substrate 20′, 20″ or 20′″ before the back sheet 12′, 12″ or 12′″ is folded. This means that the seal 48′, 48″ or 48′″ also extends completely around the perimeter, 26′, 26″ or 26′″ respectively, of the liquid permeable substrate 20′, 20″ or 20′″ after the back sheet 12′, 12″ or 12′″ is folded. Like the embodiment shown in FIGS. 1-4, the seal 48′ or 48″ also functions to seal the composition or formulation 46′ or 46″ from outside air.

Referring to FIGS. 5, 6, 8 and 9, a pocket 64′ or 64″ can optionally be formed on the outer surface 16′ or 16″ of the back sheet, 12′ or 12″ respectively. In FIGS. 5 and 6, the pocket 64′ is formed by folding the outer surface 16′ of the back sheet 12′ upon itself. The side edges 60′ and 62′ of the back sheet 12′ can be secured or attached to the outer surface 16′ of the back sheet 12′ by an adhesive or by an ultrasonic bond. In FIGS. 8 and 9, an alternative method of forming the pocket 64″ is shown wherein a separate piece of material 68″ is attached or secured onto the outer surface 16″ of the back sheet 12″. In either embodiment, the pockets 64′ and 64″ are sized to allow a user to insert one or more of his or her fingers or thumb from one hand into it so as to facilitate moving or wiping the substrate 20′ or 20″ over an exposed surface. Desirably, the pocket 64′ or 64″ will be sized to easily receive and snugly hold two or three fingers and/or a thumb of the user's hand. When the pocket 64′ or 64″ is able to snugly encompass two, three or even four fingers and/or a thumb of the user's hand, the disposable applicator 10, 10′ or 10″ can be easily moved back and forth, in a circular motion or in some other motion, on the exposed surface, i.e. a human skin, without the fingers and/or thumb dislodging from the pocket 64′ or 64″. This feature increases the usefulness of the disposable applicator 10, 10′ or 10″.

Referring again to FIGS. 5, 9 and 10, the disposable applicators 10′ and 10″ further include an advertisement 66′ or 66″. The advertisement 66′ or 66″ can be printed, embossed or otherwise formed onto the outer surface 16′ or 16″ of the back sheet 12′ or 12″, respectively. Alternatively, the advertisement 66′ or 66″ can be in the form of a label or patch that is attached to the outer surface 16′ or 16″ of the respective back sheet 12′ or 12″. The advertisement 66′ or 66″ can function to provide information to the user. Such information can include the name of the manufacturer, the trademark of the manufacturer and/or the brand name of the product. In addition, the advertisement 66′ or 66″ can provide instructions as to how to use the product and/or safety information for the user. Furthermore, the advertisement 66′ or 66″ can provide instructions as to how to properly dispose of the disposable applicator 10′ or 10″, etc.

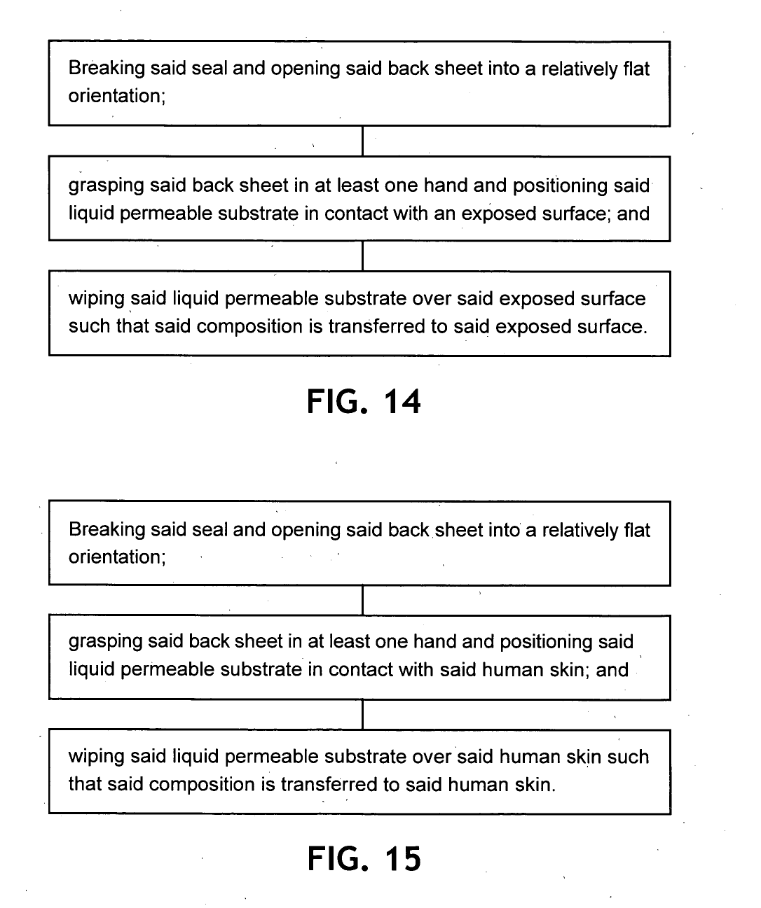

Turning now to FIG. 14, a flow diagram is shown which depicts a method of using the disposable applicator 10, 10′ or 10″. The method includes breaking the seal 48, 48′ or 48″ by pulling on the opposite ends 30 and 32, 30′ and 32′, and 30″ and 32″, of the respective back sheet 12, 12′ or 12″. By simultaneously applying a separating force to both halves of the backing sheet 12, 12′ or 12″, the seal 48, 48′ or 48″ can be broken without tearing or destroying the material from which the backing sheet 12, 12′ or 12″ is constructed. The amount of separation force needed to open the seal 48, 48′ or 48″ can be adjusted during manufacture of the disposable applicator 10, 10′ or 10″. The force required to open the seal 48, 48′ or 48″ should not be so great that it causes difficulty for the end user to open the disposable applicator 10, 10′ or 10″.

The back sheet 12, 12′ or 12″ is then opened into a relatively flat or planar orientation. This flat or planar orientation is comparable to the orientation which the back sheet 12, 12′ or 12″ exhibited before it was folded. For the embodiment shown in FIGS. 1-4, the user would align one or more of his or her fingers and/or thumb, desirably three fingers, onto the outer surface 16 of the back sheet 12. The user would also position his or her thumb and little finger onto the inner surface 14 of the back sheet 12. By doing so, the user would be able to easily grasp the disposable applicator 10 and move it back and forth, in a circular motion, or in some other motion, on the exposed surface.

For the two embodiments shown in FIGS. 5 and 6 and 7-10, respectively, the user would be able to insert one, two or three of his or her fingers into the pocket 64′ or 64″ after the breakable seal 48′ or 48″ has been broken and the back sheet 12′ or 12″, respectively, has been opened into a flat or planar orientation. The user would then position his or her thumb and little finger onto the opposite sides of the inner surface 14′ or 14″ of the back sheet 12′ or 12″, respectively. Alternatively, the user could insert four of his or her fingers into the pocket 64′ or 64″ and position his or her thumb onto one side of the inner surface 14′ or 14″ of the back sheet 12′ or 12″, respectively. By doing so, the user would be able to easily grasp the disposable applicator 10′ or 10″ and move it back and forth, in a circular motion, or in some other motion, on the exposed surface. The wiping motion will allow the composition or formulation 46′ or 46″ to be transferred from the liquid permeable substrate 20′ or 20″ onto said exposed surface. Desirably, the exposed surface is human skin and the composition or formulation 46′ or 46″ is medication in the form of an ointment, salve or cream that can be applied to the skin to sooth and/or heal it.

Referring now to FIG. 15, a flow diagram is shown which depicts a method of applying a composition or formulation 46, 46′ or 46″ onto a human's skin using the disposable applicator 10, 10′ or 10″, respectively. This method includes breaking the seal 48, 48′ or 48″ and opening the back sheet 12, 12′ or 12″, respectively, into a relatively flat or planar orientation. The user would then grasp the back sheet 12, 12′ or 12″ in one hand using the technique described above, which depends on the design and construction of the disposable applicator 10, 10′ or 10″. The liquid permeable substrate 20, 20′ or 20″ is placed in contact with the human skin and is wiped thereon. The wiping action can include a back and forth motion, a circular motion or some other motion as determined by the user. As the liquid permeable substrate 20, 20′ or 20″ is wiped over the human skin, the composition or formulation 46, 46′ or 46″ will be transferred to the human skin. An even or uniform distribution of the composition or formulation 46, 46′ or 46″ onto the skin can be obtained in this manner.

While the invention has been described in conjunction with several specific embodiments, it is to be understood that many alternatives, modifications and variations will be apparent to those skilled in the art in light of the aforegoing description. Accordingly, this invention is intended to embrace all such alternatives, modifications and variations that fall within the spirit and scope of the appended claims.

We claim:

1. A method of making a disposable applicator comprising the steps of:
a) forming a back sheet having an inner surface, an outer surface and a perimeter;
b) forming a liquid permeable substrate having a first surface, a second surface and a perimeter;
c) securing said first surface of said liquid permeable substrate to said inner surface of said back sheet;
d) applying a composition to said liquid permeable substrate, said composition capable of being transferred to an exposed surface by wiping said liquid permeable substrate against said exposed surface;
e) forming a seal on said inner surface of said back sheet; and
f) folding said back sheet on itself such that said seal extends completely around said perimeter of said substrate to protect said composition.

2. The method of claim 1 wherein said back sheet is formed from a vapor-impermeable material.

3. The method of claim 1 wherein said back sheet is formed from a moisture-impermeable material.

4. The method of claim 1 wherein said back sheet is formed from a liquid-impermeable material.

5. The method of claim 1 wherein said seal is formed before said composition is applied to said liquid permeable substrate.

6. The method of claim 1 wherein said back sheet has a transverse axis and said back sheet is folded along said transverse axis.

7. The method of claim 6 wherein a chamber is formed within an area encompassed by said seal once said back sheet is folded along said transverse axis.

8. The method of claim 1 wherein said back sheet is formed from a film.

9. The method of claim 8 wherein said film is a polyolefin film.

10. A method of using a disposable applicator formed of a back sheet, a liquid permeable substrate, a composition and a seal, said back sheet having an inner surface, an outer surface and a perimeter; said liquid permeable substrate having a first surface, a second surface and a perimeter, and said first surface being secured to said inner surface of said back sheet such that said inner surface extends around said perimeter of said substrate; said composition applied to said liquid permeable substrate; and said seal is formed on said inner surface of said back sheet and said back sheet is folded upon itself such that said seal extends completely around said perimeter of said substrate to protect said composition, said method comprising the steps of:
a) breaking said seal and opening said back sheet into a relatively flat orientation;
b) grasping said back sheet in at least one hand and positioning said liquid permeable substrate in contact with an exposed surface; and
c) wiping said liquid permeable substrate over said exposed surface such that said composition is transferred to said exposed surface.

11. The method of claim 10 wherein a pocket is formed on said back sheet by folding said back sheet upon itself, said pocket being sized to receive at least one digit of a user, and after said seal is broken, said user inserts a digit into said pocket so as to be able to wipe said liquid permeable substrate over said exposed surface.

12. The method of claim 11 wherein said back sheet is secured to itself by an adhesive to form said pocket.

13. The method of claim 11 wherein said back sheet is secured to itself by an ultrasonic bond to form said pocket.

14. The method of claim 10 wherein a pocket is formed on said back sheet by securing a separate piece of material to said back sheet, said pocket being sized to receive at least one finger of a user.

15. The method of claim 10 wherein said wiping is accomplished using an applied force ranging from between about 0.01 psi to about 5 psi.

16. A method of applying a composition onto a human's skin using a disposable applicator including a back sheet having an inner surface, an outer surface and a perimeter; a liquid permeable substrate having a first surface, a second surface and a perimeter, and said first surface being secured to said inner surface of said back sheet; a composition applied to said liquid permeable substrate, and a seal formed on said inner surface of said back sheet and said back sheet is folded upon itself such that said seal extends completely around said perimeter of said substrate to protect said composition, said method comprising the steps of:
a) breaking said seal and opening said back sheet into a relatively flat orientation;
b) grasping said back sheet with one hand and positioning said liquid permeable substrate in contact with said human skin; and
c) wiping said liquid permeable substrate over said human skin such that said composition is transferred to said human skin.

17. The method of claim 16 wherein said wiping is accomplished using an applied force ranging from between about 0.01 psi to about 5 psi.

18. The method of claim 17 wherein said applied force ranges from between about 0.1 psi to about 3 psi.

19. The method of claim 18 wherein said applied force ranges from between about 0.2 psi to about 2 psi.

20. The method of claim 16 wherein said outer surface of said back sheet contains a pocket which is sized to receive at least two fingers of a user, and after said seal is broken, said user inserts at least two fingers into said pocket so as to be able to wipe said liquid permeable substrate over said human skin.

21. A method of making a disposable applicator comprising the steps of:
a) forming a back sheet having an inner surface, an outer surface and a perimeter;
b) forming a liquid permeable substrate having a first surface, a second surface and a perimeter;
c) securing said first surface of said liquid permeable substrate to said inner surface of said back sheet;
d) applying a composition to said liquid permeable substrate, said composition capable of being transferred to an exposed surface by wiping said liquid permeable substrate against said exposed surface;
e) folding said back sheet on itself; and
f) forming a seal around said perimeter of said substrate to protect said composition.
